(19) United States
(12) Patent Application Publication
(10) Pub. No.: US 2010/0021380 A1
(43) Pub. Date: Jan. 28, 2010

(54) DIEGHYLENETRIAMINEPENTAACETIC ACID (DTPA)-MODIFIED FERROFLUID, PREPARATION METHOD OF THE SAME AND USES OF THE SAME IN PREPARATION OF PEPTIDE FERROFLUID

(75) Inventors: JEN-CHIEH CHUNG, Taoyuan County (TW); KUNG-TIEN LIU, Taoyuan County (TW); MIN-NAN CHEN, Taoyuan County (TW); FU-DER MAI, Taoyuan County (TW); CHUN-CHAO CHANG, Taoyuan County (TW)

Correspondence Address: WPAT, PC;INTELLECTUAL PROPERTY ATTORNEYS, 7225 BEVERLY ST., ANNANDALE, VA, 22003, US

(73) Assignee: Institute of Nuclear Energy Research Atomic Energy Council, Executive Yuan, Taoyuan County (TW)

(21) Appl. No.: 12/370,978

(22) Filed: Feb. 13, 2009

(30) Foreign Application Priority Data
Jul. 25, 2008 (TW) 097128454

(51) Int. Cl. A61K 51/04; A61K 38/16; A61K 31/197; A61K 9/14; A61K 38/02; A61K 33/26

(52) U.S. Cl. 424/1.65; 514/6; 514/566; 424/489; 424/647

(57) ABSTRACT

A present invention relate to a diethylenetriaminepentaacetic acid (DTPA)-modified ferrofluid and a preparation method of the same. The DTPA-ferrofluid contains DTPA and a nano ferrofluid. The DTPA-ferrofluid can be further mixed with a peptide. Unmodified or modified peptide ferrofluids prepared from the DTPA-modified ferrofluid, such as unmodified or modified octreotide-containing or unmodified or modified lanreotide-containing ferrofluid.

### BACKGROUND OF THE INVENTION

1. Field of the Invention

The present invention relates to a diethylenetriaminepenta acetic acid (DTPA)-modified ferrofluid (referred to as DTPA-ferrofluid hereinafter). The DTPA-ferrofluid contains DTPA and a nano ferrofluid. The DTPA-ferrofluid can be further mixed with a peptide (for example, peptide for cancer cell localization), to prepare a peptide ferrofluid having particular uses (for example, medical use).

2. Related Art

Magnetic materials have been widely used in manufacture of magnetic memory materials, such as recording tape, disc, and magnetic tape, in construction materials, such as printing ink, paint, and coating, and in mechanical uses, such as electromagnetic switching and shaft sealing. Recently, as continuous innovations in preparation method have been made by scientists, many new application fields are also gradually developed, and great interest are aroused, for example, biomedical applications, such as purification of medicine, protein, and DNA, and treatment of environment wastes, for example, a magnetic material with a particle diameter of less than 1000 nm prepared by mixing magnetic particles into a polar solution of carbohydrate, which is useful in cell isolation and purification [see, for example, U.S. Pat. No. 4,687,748 (1987)]. The isolation technology utilizing magnetism can be divided into two type according to the properties of the material to be treated: (1) isolation of inherently magnetic materials with an externally applied magnetic field; and (2) isolation of a non-magnetic material by reacting with a magnetic material to combine the two together, and then isolating with an externally applied magnetic field. In order to efficiently combine the non-magnetic material and the magnetic material, species and preparation methods of different magnetic materials will play an important role.

For magnetic materials, preparation methods vary with different application objects and requirements, and the most common ones include (1) mechanical grinding, for example, mixing substances, for example an organic carrier, such as a glycol and an ester, magnetic particles and a cationic surfactant for mechanical grinding, to prepare a ferrofluid to improve the conductivity and sealing effect of the magnetic disc design of a computer (see, for example, U.S. Pat. No. 4,604,222 (1986)); (2) oxidation, for example, reacting a ferrous solution with a phosphate, such as sodium orthophosphate, and a basic hydroxide, to generate ferrous hydroxide (II), and then introducing oxygen for oxidation, to generate a magnetic ferrite powder (see, for example, U.S. Pat. No. 6,140,001 (2000)); and (3) chemical co-precipitation, for example, mixing a magnetic iron powder, such as ZnMn ferrite and NiZn ferrite with conductive particles, such as gold, silver, copper, aluminum, and graphite, to get a ferrofluid useful in the application of electromagnetic valve switching (U.S. Pat. No. 6,743,371 (2004)). Because the magnetic particles themselves will attract each other and get aggregated, particle surface treatment is required in the preparation process, such that particles can be effectively isolated from each other, to obtain a powder with small particle diameter, thus being more easily dispersed in a solvent into a fluid form. Moreover, in order to make the prepared ferrofluid have lipophilic or hydrophilic property, the surface treatment manners are generally different.

For preparation of an oil-based ferrofluid having lipophilic property, the ferrofluid can be prepared by adding an organic dispersant containing a hydrophilic group into an organic solvent having a low melting point, and dispersing magnetic particles into the mixture, and then removing the organic solvent having a low melting point through evaporation. The ferrofluid thus prepared is useful in seal design of vacuum instruments (U.S. Pat. No. 5,124,060 (1992)). For example, an oil-based ferrofluid can be prepared by directly mixing α-Fe₂O₃ powder, an oil (Ampro Type II oil), and a surfactant, such as polyolefin anhydride to form a slurry, and then grinding (see, for example, U.S. Pat. No. 6,068,785 (2000)). Therefore, applications of oil-based ferrofluids in common people's livelihood industry are mainly found in, for example, magnetic memory materials, mechanical seal design, or treatment of metal ions in inorganic waste water, and removal of floating oil or trace organic components in water, while applications in organisms are still under study. Further, preparation methods of oil-based ferrofluids are mainly mechanical grinding, which will decrease the binding force for attaching oil and surfactant to the surface of magnetic particles, and thus the surface binding substance may easily fall off, thereby affecting utilization efficiency. Preparation and applications of ferrofluid having hydrophilic property are similar to those of oil-based ferrofluids, and have some disadvantages and need to be improved.

Medical peptides are, for example, somatostatin analogues, such as lanreotide and octreotide. Lanreotide is a first slow-release somatostatin analogue used in treatment of clinical symptoms of acromegaly and carcinoid tumor, and octreotide has a structure comprising 8 amino acids of the formula below:

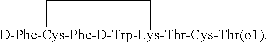

Like somatostatin analogues, octreotide binds to receptors on the surface of cancer cells, and has function of inhibiting the growth rate of cancer cells, thus arousing great interest in medical field and is used to carry out various clinical application researches. Preparation of octreotide has been reported in many relevant literatures and patents, including, for example, liquid phase synthesis (see, for example, U.S. Pat. No. 4,395,403 (1983), U.S. Pat. No. 6,987,167(2006)) and solid phase synthesis (see, for example, U.S. Pat. No. 5,889,146 (1999), U.S. Pat. No. 6,476,186 (2002), and U.S. Pat. No. 6,346,601 (2002)), in which U.S. Pat. No. 6,987,167 has disclosed production procedures and methods for preparing commercialized large-scale (kilogram level) octreotide (I) with a yield of 80%-90%. Recently, octreotide is also labeled with a radioisotope (e.g. In¹¹¹, Y⁹⁰), and used in tumor diagnosis in nuclear medicine [see, for example, U.S. Pat. No. 7,045,503 (2006)]. An octreotide labeled with In¹¹¹, ¹¹¹In-DTPA-D-Phe¹-octreotide or ¹¹¹In-OctreoScan®, has been approved and marketed in America and European, and is used for development of neuroendocrine tumors. In addition to In¹¹¹, researches with other different isotope labels are also carried out (e.g. ^{99m}Tc[N4(D)Ph¹]-octeotide) (Maina et al, Journal of Nuclear Biology and Medicine, p 452, 1994) and [⁹⁰Y-DOTA-Dphe1,Tyr3] octreotide (⁹⁰Y-SMT 487) (Stolz, et al; European Jnl. Of Nucl. Med.,25(7), 668, 1998).

Literatures disclose that many researches on octreotide have been carried out, but the researches mainly focus on synthesis process, medical detection, and effect on inhibiting the growth rate of cancer cells, few application researches of therapeutic effect is performed, and except for the preparation and application in combination with isotope, combination research with other non-radioactive isotope is also fewer.

Diethylenetriaminepenta acetic acid (DTPA) is a chelating agent for calcium salts or zinc salts, and is generally used to treat a patient suffering from internal contamination of some radioactive substance in prior art, but the combination uses with ferrofluids have not been disclosed.

### SUMMARY OF THE INVENTION

Accordingly, the present invention is directed to a diethylenetriaminepenta acetatic acid (DTPA)-modified ferrofluid (referred to as DTPA-ferrofluid hereinafter). The DTPA-ferrofluid can further combine a peptide, such as unmodified or modified lanreotide or octreotide, to obtain a peptide-containing DTPA-ferrofluid. Cancer cell localization can be achieved by injecting the prepared octreotide ferrofluid into an organism, and-high temperature treatment or therapeutic purpose can be achieved by conveniently utilizing an externally applied magnetic field adjuvant with high-frequency wave to cause heat generation. The subject of the present invention has the advantages of improving therapeutic effect with simple equipment and easy operation.

According to a first aspect of the present invention, a DTPA-modified ferrofluid is provided, containing (a) a nano ferrofluid; and (b) DTPA, in which the molar ratio of the nano ferrofluid to DTPA is 90% to 110%.

According to a second aspect of the present invention, a peptide ferrofluid is provided, containing: (1) a DTPA-modified ferrofluid, containing (a) a nano ferrofluid; and (b) DTPA, in which the molar ratio of the nano ferrofluid to DTPA is 90% to 110%; and (2) a peptide, selected from a group consisting of an unmodified peptide and a modified peptide.

According to a third aspect of the present invention, a method for preparing a DTPA-modified ferrofluid is provided, including: (a) mixing an aqueous solution of a magnetic compound with DTPA to form a mixture; (b) adding a basic solution to the mixture; (c) taking a precipitate out from the mixture; and (d) lyophilizing the precipitate, to form a lyophilized finished product.

According to a fourth aspect of the present invention, a method for preparing a DTPA-modified ferrofluid is provided, in which the ferrofluid contains a magnetic compound such as ferroferric oxide (Fe₃O₄). The method includes: (a) dissolving 3 g to 5 g of ferrous chloride (FeCl₂) hydrates with 3 to 5 hydration water molecules, and 10 g to 13 g of ferric chloride (FeCl₃) hydrates with 5 to 7 hydration water molecules into 100 ml to 200 ml of deoxygenated water, to form a pre-mixture; (b) refluxing and heating at 80° C. to 90° C., and adding 10 ml to 20 ml of 20% to 25% ammonium hydroxide when the temperature of the pre-mixture is raised to 85° C.; (c) taking a precipitate out from the pre-mixture, and adding glycerol to the precipitate; (d) adding the precipitate containing glycerol into a solution having a pH of 3 to 5 of N-(2-aminoethyl)-3-aminopropyl-trimethoxysilane (APTES) in deoxygenated water, to form a mixture, and then refluxing and heating the mixture at 80° C. to 100° C. for 2.5 h to 3.5 h; (e) cooling the mixture to room temperature, adding 2 g to 4 g of DTPA, and then refluxing and heating at boiling temperature for 2.5 h to 3.5 h; and (f) cooling the mixture to room temperature, and lyophilizing to get a lyophilized semi-finished product.

According to a fifth aspect of the present invention, a method for preparing a DTPA-modified ferrofluid containing urea is provided, including: preparing a solution containing a DTPA-modified ferrofluid; and mixing the solution with urea, in which the DTPA-modified ferrofluid contains (a) a nano ferrofluid and (b) DTPA at a molar ratio of 90% to 110%.

According to a sixth aspect of the present invention, a method for preparing a peptide ferrofluid is provided, including: (a) preparing a DTPA-modified ferrofluid, which contains (1) a nano ferrofluid and (2) DTPA at a molar ratio of 90% to 110%; (b) adding a peptide to form a peptide ferrofluid pre-product; and (c) dispersing the peptide ferrofluid pre-product. Especially, the molar ratio of the DTPA-modified ferrofluid to the peptide is greater than 1.

These and other aspects and features of the present invention will be fully understood, when reading the following detailed description with reference to accompanied drawings.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given herein below for illustration only, and thus are not limitative of the present invention, and wherein:

FIG. 1 is a synthesis flow chart of a DTPA-modified ferrofluid (DTPA-γ-Fe₂O₃); and

FIG. 2 is a synthesis flow chart of a DTPA-modified ferrofluid (DTPA-Fe₃O₄).

### DETAILED DESCRIPTION OF THE INVENTION

According to a first preferred embodiment of the present invention, a diethylenetriaminepenta acetic acid (DTPA)-modified ferrofluid contains (a) a nano ferrofluid, and (b) DTPA, in which the molar ratio of the nano ferrofluid to DTPA is 90% to 110%. Preferably, the molar ratio of the nano ferrofluid to DTPAis 100%.

Preferably, the nano ferrofluid has a particle diamteter ranging from 20 nm to 150 nm, and more preferably from 60 nm to 100 nm.

In a non-limitative example, the nano ferrofluid can be prepared from a magnetic compound, such as γ-ferric oxide (γ-Fe₂O₃) or ferroferric oxide (Fe₃O₄).

In a further preferred embodiment, the DTPA-modified ferrofluid further contains a radioisotope selected from a group consisting of yttrium-90, rhenium-188, indium-111, gadolinium-67, and the like.

According to a second preferred embodiment of the present invention, a peptide ferrofluid contains (1) a DTPA-modified ferrofluid, containing (a) a nano ferrofluid, and (b) DTPA, in which the molar ratio of the nano ferrofluid to DTPA is 90% to 110%; and (2) a peptide, selected from a group consisting of an unmodified peptide and a modified peptide. Preferably, the molar ratio of the nano ferrofluid to DTPA is 100%, and preferably the molar ratio of the DTPA-modified ferrofluid to the peptide is greater than 1.

Preferably, the nano ferrofluid has a particle diameter ranging from 20 nm to 150 nm, and more preferably from 60 nm to 100 nm.

In a non-limitative example, the nano ferrofluid can be prepared from a magnetic compound, such as γ-ferric oxide (γ-Fe₂O₃) or ferroferric oxide (Fe₃O₄).

Preferably, the unmodified peptide is selected from a group consisting of octreotide and lanreotide, and preferably, the modified peptide is selected from a group consisting of DTPA-modified octreotide and DTPA-modified lanreotide modified.

In a further preferred embodiment, the peptide ferrofluid further contains a radioisotope, selected from a group consisting of yttrium-90, rhenium-188, indium-111, gadolinium-67, and the like.

In a still further preferred embodiment, the DTPA-modified ferrofluid further contains urea.

According to a third preferred embodiment of the present invention, a method for preparing a DTPA-modified ferrofluid includes: (a) mixing an aqueous solution of a magnetic compound with DTPA, to form a mixture; (b) adding a basic solution to the mixture; (c) taking a precipitate out from the mixture; and (d) lyophilizing the precipitate, to form a lyophilized finished product. Preferably, the magnetic compound is γ-ferric oxide (γ-Fe₂O₃).

Preferably, step (a) includes: (a1) dissolving 4 g to 6 g of ferrous sulfate (FeSO₄) hydrates with 6 to 8 hydration water molecules into 40 ml to 60 ml of water; (a2) adding 3 g to 5 g of DTPA; and (a3) refluxing and heating at 80° C. to 100° C., and preferably 90° C. for 20 min to 1.5 h, preferably 20 min to 40 min, and more preferably 30 min.

Preferably, step (b) includes: slowly dripping 8 ml to 12 ml of a 10% to 40% sodium hydroxide solution, and preferably a 30% sodium hydroxide solution, and refluxing for 1.5 h to 2.5 h, and preferably 2 h.

Preferably, step (c) includes: taking the precipitate out by attracting with magnetic force and pouring. More preferably, step (c) further includes washing the precipitate at least 3 times with water and acetone.

Preferably, step (d) further includes a step of drying the precipitate with IR, before lyophilizing the precipitate.

According to a fourth preferred embodiment of the present invention, a method for preparing a DTPA-modified ferrofluid containing a magnetic compound such as ferroferric oxide (Fe₃O₄), includes: (a) dissolving 3 g to 5 g of ferrous chloride (FeCl₂) hydrates with 3 to 5 hydration water molecules, and 10 g to 13 g of ferric chloride (FeCl₃) hydrates with 5 to 7 hydration water molecules into 100 ml to 200 ml of deoxygenated water, to form a pre-mixture; (b) refluxing and heating at 80° C. to 100° C., and adding 10 ml to 20 ml 20% to 25% ammonium hydroxide when the temperature of the pre-mixture is raised to 85° C.; (c) taking a precipitate out from the pre-mixture, and adding glycerol to the precipitate; (d) adding the precipitate containing glycerol into a solution having a pH of 3 to 5 of N-(2-aminoethyl)-3-aminopropyl-trimethoxysilane (APTES) in deoxygenated water, to form a mixture, and then refluxing and heating the mixture at 80° C. to 100° C. for 2.5 to 3.5 h; (e) cooling the mixture to room temperature, adding 2 g to 4 g of DTPA, and then refluxing and heating at boiling temperature for 2.5 h to 3.5 h; and (f) cooling the mixture to room temperature, and lyophilizing to get a lyophilized semi-finished product.

According to a fifth preferred embodiment of the present invention, a method for preparing a DTPA-modified ferrofluid containing urea includes: preparing a solution containing a DTPA-modified ferrofluid; and mixing the solution with urea. Preferably, the DTPA-modified ferrofluid contains (a) a nano ferrofluid and (b) DTPA at a molar ratio of 90% to 110%.

In a non-limitative example, the nano ferrofluid can be prepared from a magnetic compound selected from γ-ferric oxide (γ-Fe₂O₃) and ferroferric oxide (Fe₃O₄).

According to a sixth preferred embodiment of the present invention, a method for preparing a peptide ferrofluid includes: (a) preparing a DTPA-modified ferrofluid, which contains (1) a nano ferrofluid and (2) DTPA at a molar ratio of 90% to 110%; (b) adding a peptide to form a peptide ferrofluid pre-product; and (c) dispersing the peptide ferrofluid pre-product. Preferably, the molar ratio of the DTPA-modified ferrofluid to the peptide is greater than 1.

In a non-limitative example, the nano ferrofluid can be prepared from a magnetic compound selected from γ-ferric oxide (γ-Fe₂O₃) and ferroferric oxide (Fe₃O₄).

Preferably, the peptide is selected from a group consisting of unmodified peptide and modified peptide. More preferably, the unmodified peptide is selected from a group consisting of octreotide and lanreotide, and more preferably, the modified peptide is selected from a group consisting of DTPA-modified octreotide and DTPA-modified lanreotide.

In a further embodiment, step (a) further includes adding urea.

Cancer cell localization can be achieved by injecting the peptide ferrofluid of the present invention, and peptide ferrofluid prepared by the method of the present invention for preparing a peptide ferrofluid, for example octreotide ferrofluid, into an organism, and high temperature treatment or therapeutic purpose can be achieved by conveniently utilizing an externally applied magnetic field adjuvant with high-frequency wave to cause heat generation. The subject of the present invention has advantages such as improving therapeutic effect with simple equipment, and easy operation.

Especially, for example, it is confirmed that cancer cells have somatostatin analogue receptors on the surface thereof, and octreotide is a somatostatin analogue and will binds to receptors on the surface of cancer cells as somatostatin. Currently, octreotide is widely used in tumor diagnosis in nuclear medicine, for example, researches concerning indium-111 octreotide tumor injection, octreoscan, and gene therapy.

Common commercial available and commercialized ferrite magnet powders, having a particle diameter not easily being controlled at nano level, and having no peptide-philic property, cannot achieve the effect of efficient dispersion, if it is directly mixed with an octreotide tumor injection, so it is required to modify the surface of the ferrite powder to have affinity to octreotide, so as to efficiently bind to the octreotide tumor injection and be dispersed therein. Nano ferrofluid prepared with a novel method in the present invention has peptide-philic property and a particle diameter ranging from about 20 nm to 150 nm, and preferably 60-100 nm, and can be uniformly mixed with an octreotide tumor injection before use, such that it becomes a magnetic fluid having property of octreotide tumor injection, without affecting the existing properties of octreotide tumor injection, for example, radioactive tag. Octreotide tumor injection ferrofluid can be localized on cancer cells, and then iron molecules are caused to generate heat (about 38° C.-50° C., and preferably 42° C.) through the oscillation effect of high-frequency magnetic field, so as to achieve the purpose of eliminating the targeted cells.

Furthermore, the method for preparing a ferrofluid according to the present invention is carried out at a low or normal temperature to generate a high magnetic ferrite powder without sintering at high temperature, and at the same time, surface modification of the high magnetic ferrite powder can be performed with a peptide-philic functional group, for example DTPA. The ferrite powder thus prepared is sterilized and packaged, and uniformly mixed with appropriate amount of medical octreotide tumor injection at a molar ratio of the ferrofluid to the octreotide tumor injection of greater than 1.0, so as to afford a mixture ready for clinical use. Simple use and easy operation are further features of the present invention.

With the peptide ferrofluid of the present invention, and the peptide ferrofluid prepared via a method of the present invention for preparing a peptide ferrofluid, the ferrofluid can be directed and controlled to be gathered at specific sites by means of an externally applied magnetic field, then heat is generated by oscillating with high frequency wave, and the temperature is raised to about 38° C.-50° C., and preferably 42° C., thus leading to apoptosis, thereby a hyperthermia treatment effect is achieved.

Moreover, the ferrofluid of the present invention and the ferrofluid prepared by a method of the present invention for preparing a ferrofluid can have radioactivity and ferromagnetism by combining a radioisotope, such as a tag, for example, indium-111, yttrium-90, gadolinium-68, and rhenium-188, or other modifications, when mixed and oscillate with a octreotide tumor injection, thus being more convenient and having more function.

Embodiments below will illustrate preparation, features, and uses of composition according to the present invention. These embodiments are not intended to limit scope of the present invention in any way. While the present invention has been described with reference to particular specific example, it is apparent to those of skill in the art that various changes and modifications can be made without deviating scope of the present invention.

### Embodiment 1

Preparation of Lyophilized Magnetic DTPA-Modified Nano γ-Ferric Oxide (γ-Fe₂O₃) Ferrofluid

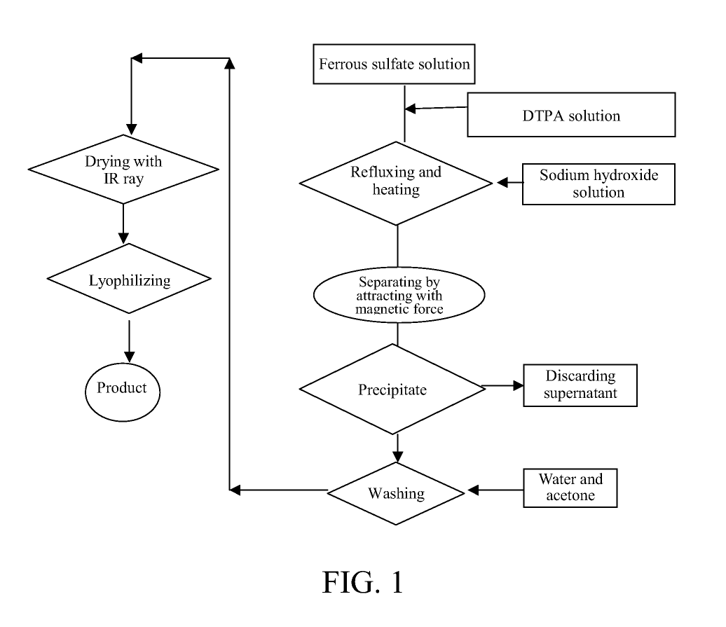

Referring to FIG. 1, this embodiment provides a method for synthesizing a ferrofluid containing DTPA and γ-Fe₂O₃, and the resulting product can be uniformly dispersed in, for example a medical octreotide tumor injection and/or other peptide solutions, such that the solution has ferromagnetism, so as to facilitate the direction and localization of an externally applied magnetic field.

4 g to 6 g, and preferably 5.66 g of ferrous sulfate (FeSO₄.7H₂O) hydrates with 6 to 8, and preferably 7 hydration water molecules was dissolved into 40 ml to 60 ml, and preferably 50 ml of water. Next, 3 g to 5g, and preferably 3.93 g of DTPA was added, and then refluxed and heated at a temperature ranging from 80° C. to 100° C., and preferably 90° C. for 20 min to 1.5 h, and preferably 30 min. Afterwards, 8 ml to 12 ml, and preferably 10 ml of a 10% to 40%, and preferably 30% sodium hydroxide solution was added dropwise slowly. Then, the solution was refluxed for 1.5 h to 2.5 h, and preferably 2 h, cooled to room temperature, washed with water 3 times, followed with acetatone 3 times by attracting with magnetic force and pouring, dried with IR light to almost complete dryness, and was lyophilized to get a lyophilized finished product. After being dissolved in water, appropriate amount of packaged lyophilized finished product, could be added to a commercial available octreotide tumor injection or a developing agent, for example, octreotide unmodified or modified with DTPA, in which the molar ratio of the lyophilized finished product to the modified or unmodified octreotide was greater than 1. Then, the solution was ultrasonated for 3 min to 30 min, and preferably 10 min, to get a ferrofluid octreotide tumor injection or developing agent.

### Embodiment 2

Preparation of γ-Ferric Oxide (γ-Fe₂O₃) Ferrofluid Containing Urea

Similar to Embodiment 1, a lyophilized magnetic DTPA-modified nano γ-Fe₂O₃ semi-finished product was first synthesized. Next, appropriate amount of the prepared lyophilized semi-finished product was added into water, and fully dispersed to get a solution. Then, the DTPA-γ-Fe₂O₃ solution thus prepared was added into equal mole of urea, and refluxed and heated at boiling temperature for 1.5 h to 2.5 h, and preferably 2 h. Afterwards, the mixture was cooled to room temperature, and washed at least 3 times with water by attracting with magnetic force and pouring, dried with an IR light to almost complete dryness, and was lyophilized, to get a lyophilized semi-finished product containing urea.

### Embodiment 3

Preparation of Octreotide Tumor Injection Ferrofluid Containing γ-Ferric Oxide (γ-Fe₂O₃)

Similar to Embodiment 2, a lyophilized magnetic DTPA-modified nano γ-Fe₂O₃ semi-finished product was first synthesized. Next, appropriate amount of the prepared lyophilized semi-finished product was added into water, and fully dispersed to get a solution. Then, the urea-DTPA-γ-Fe₂O₃ solution thus prepared was added into a commercial available octreotide tumor injection or a developing agent, for example, octreotide unmodified or modified with DTPA, in which the molar ratio of the lyophilized finished product to the unmodified or modified octreotide was greater than 1.0. Then, the solution was ultrasonated for 3 min to 30 min, and preferably 10 min, to get a ferrofluid octreotide tumor injection or developing agent. The lyophilized finished product could also be added into a solution labeled with a radioisotope, and then ultrasonated for 3 min to 15 min, and preferably 10 min, to get a ferrofluid octreotide tumor injection or developing agent.

### Embodiment 4

Preparation of Lyophilized DTPA-Modified Nano Ferroferric Oxide (Fe₃O₄) Ferrofluid

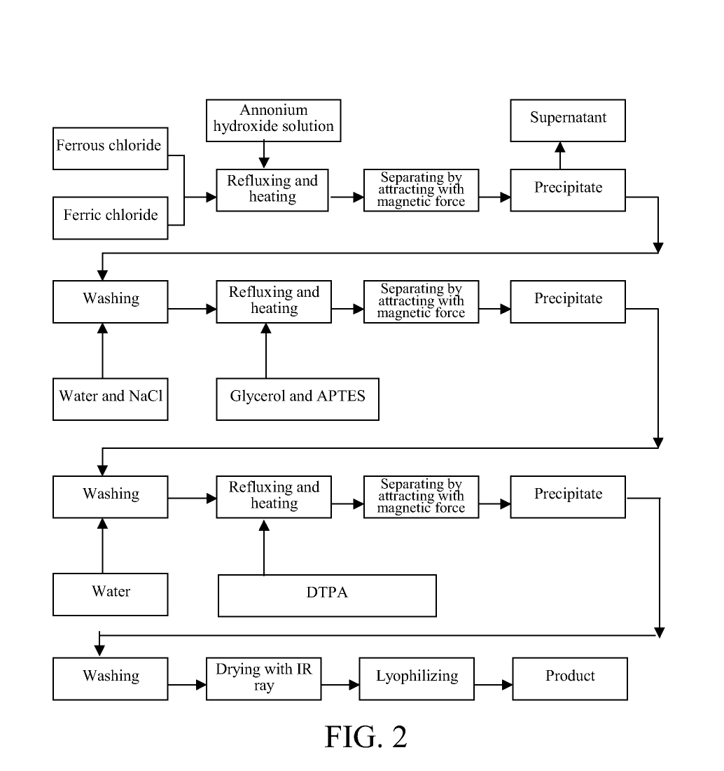

Referring to FIG. 2, this embodiment provides a method for synthesizing a ferrofluid containing DTPA and Fe₃O₄, and the resulting product can be uniformly dispersed into, for example, a medical octreotide tumor injection and/or other peptide solutions, such that the solution has ferromagnetism, thus being convenient for the direction and localization of an externally applied magnetic field.

Ferrous chloride tetrahydrate (FeCl₂.4H₂O) of 4.302 g and ferric chloride hexahydrate (FeCl₃.6H₂O) of 11.826 g were dissolved into 200 ml of deoxygenated water bubbled with nitrogen, and then refluxed and heated at a temperature ranging from 80° C. to 100° C., and preferably 90° C. 15 ml of 25% ammonium hydroxide was added when the temperature of the pre-mixture reached 85° C. Then, refluxing and heating was continued for another 25 min to 35 min, and preferably 30 min. Afterwards, the mixture was cooled to room temperature, and washed with deoxygenated water 3 times, then 0.02 M NaCl solution 1 time, and finally deoxygenated water 1 time by attracting with magnetic force and pouring. A precipitate was collected, and 150 ml of glycerol was added to the precipitate. Furthermore, 4.0 ml of N-(2-aminoethyl)-3-aminopropyl-trimethoxysilane (APTES) was added into 30 ml of deoxygenated water, the pH was adjusted to pH 4.09 with glacial acetic acid, and finally supplemented to 40 ml by adding water. Next, the APTES solution and the precipitate solution were placed into a reactor together and refluxed for 2.5 h to 3.5 h, and preferably 3 h, at 80° C. to 100° C., and preferably 90° C. Afterwards, the mixture was cooled to room temperature and washed 3 times with deoxygenated water by attracting with magnetic force and pouring. Then, 3.93 g of DTPA was added into the reactor, refluxed at boiling temperature for 2.5 to 3.5 h, and preferably 3 h, cooling to room temperature, washed with deoxygenated water (3×) and then acetone (3×) by attracting with magnetic force and pouring, dried with IR light to almost complete dryness, and was lyophilized, to get a lyophilized semi-finished product.

### Embodiment 5

Preparation of Ferroferric Oxide (Fe₃O₄) Ferrofluid Containing Urea

Similar to Embodiment 4, a lyophilized magnetic DTPA-modified nano Fe₃O₄ semi-finished product was first synthesized. Next, appropriate amount of the prepared lyophilized semi-finished product was added into water, and fully dispersed, to get a solution. Then the DTPA-Fe₃O₄ solution thus prepared was added into 600 mg of urea, and refluxed and heated at boiling temperature for 1.5 h to 2.5 h, and preferably 2 h. Afterwards, the mixture was cooled to room temperature, and washed at least 3 times with water by attracting with magnetic force and pouring, dried with an IR light to almost complete dryness, and was lyophilized, to get a lyophilized semi-finished product containing urea.

### Embodiment 6

Preparation of Octreotide Tumor Injection Ferrofluid of Modified Nano Ferroferric Oxide (Fe₃O₄) Containing Urea

Similar to Embodiment 4, preferably, ferrous chloride tetrahydrate (FeCl₂.4H₂O) of 1.2 g and ferric chloride hexahydrate (FeCl₃.6H₂O) of 3.24 g were dissolved into 20 ml of deoxygenated water bubbled with nitrogen. Next, 50 ml of an aqueous solution containing 240 mg of DTPA was added, and then refluxed and heated at a temperature ranging from 80° C. to 100° C., and preferably 90° C. 50 ml of 1 M aqueous ammonia was slowly dripped when the temperature reached to 85° C. Then, refluxing and heating was continued for another 25 min to 35 min, and preferably 30 min. Afterwards, the mixture was cooled to room temperature, and washed with deoxygenated water 3 times, and then acetone and ethanol 2 time each by attracting with magnetic force and pouring. Then, 100 ml of deoxygenated water and 600 mg of urea were added, and refluxed and heated at boiling temperature for 1.5 h to 2.5 h, and preferably 2 h after being dissolved. Afterwards, the mixture was cooled to room temperature, and washed with deoxygenated water 3 times, then acetone and ethanol 2 time each by attracting with magnetic force and pouring, dried with IR light to almost complete dryness, and was lyophilized, to get a lyophilized semi-finished product. When being used, the product was dissolved into appropriate amount of water and then added into a commercial available octreotide tumor injection, in which the molar ratio of the lyophilized semi-finished product to the modified or unmodified octreotide was greater than 1.0. Then, the product was ultrasonated for 3 min to 30 min, and preferably 10 min, to get a ferrofluid octreotide tumor injection. The solution of the lyophilized solution can also be transferred into an octreotide tumor injection labeled with a radioisotope, and then ultrasonated for 3 min to 15 min, and preferably 10 min, to get a radiolabelled ferrofluid octreotide tumor injection or developing agent.

It should be understood that embodiments and specific examples disclosed in the present invention are merely intended to exemplify and illustrate the present invention, and imply various modifications or changes of the specification to those skilled in the art, and the modifications or changes fall into the spirit and scope of the application and the scope of accompanying Claims.

What is claimed is:

1. A diethylenetriaminepenta acetic acid (DTPA)-modified ferrofluid, comprising (a) a nano ferrofluid, and (b) DTPA, wherein a molar ratio of the nano ferrofluid to DTPA is 90% to 110%.

2. The DTPA-modified ferrofluid according to claim 1, wherein the molar ratio of the nano ferrofluid to DTPA is 100%.

3. The DTPA-modified ferrofluid according to claim 1, wherein the nano ferrofluid has a particle diameter ranging from 20 nm to 150 nm.

4. The DTPA-modified ferrofluid according to claim 3, wherein the nano ferrofluid has a particle diameter ranging from 60 nm to 100 nm.

5. The DTPA-modified ferrofluid according to claim 1, wherein the nano ferrofluid is prepared from a magnetic compound selected from γ-ferric oxide (γ-Fe₂O₃) and ferroferric oxide (Fe₃O₄).

6. The DTPA-modified ferrofluid according to claim 1, further comprising a radioisotope, selected from a group consisting of yttrium-90, rhenium- 188, indium-111, gadolinium-67, and the like.

7. A peptide ferrofluid, comprising:
(1) a diethylenetriaminepenta acetic acid (DTPA)-modified ferrofluid, comprising (a) a nano ferrofluid, and (b) DTPA, wherein the molar ratio of the nano ferrofluid to DTPA is 90% to 110%; and
(2) a peptide, selected from a group consisting of an unmodified peptide and a modified peptide.

8. The peptide ferrofluid according to claim 7, wherein the molar ratio of the nano ferrofluid to DTPA is 100%.

9. The peptide ferrofluid according to claim 7, wherein the nano ferrofluid has a particle diameter ranging from 20 nm to 150 nm.

10. The peptide ferrofluid according to claim 7, wherein the nano ferrofluid is prepared from a magnetic compound selected from γ-ferric oxide (γ-Fe₂O₃) and ferroferric oxide (Fe₃O₄).

11. The peptide ferrofluid according to claim 7, further comprising a radioisotope, selected from a group consisting of yttrium-90, rhenium-188, indium-111, gadolinium-67, and a like.

12. The peptide ferrofluid according to claim 7, wherein the unmodified peptide is selected from a group consisting of octreotide and lanreotide.

13. The peptide ferrofluid according to claim 7, wherein the modified peptide is selected from a group consisting of DTPA-modified octreotide and DTPA-modified lanreotide.

14. The peptide ferrofluid according to claim 7, wherein the molar ratio of the DTPA-modified ferrofluid to the peptide is greater than 1.0.

15. The peptide ferrofluid according to claim 14, wherein the DTPA-modified ferrofluid further comprises urea.

16. A method for preparing a diethylenetriaminepenta acetic acid (DTPA)-modified ferrofluid, comprising:
(a) mixing an aqueous solution of a magnetic compound with DTPA to form a mixture;
(b) adding a basic solution into the mixture;
(c) taking a precipitate out from the mixture; and
(d) lyophilizing the precipitate, to form a lyophilized finished product.

17. The method for preparing a DTPa-modified ferrofluid according to claim 16, wherein the magnetic compound is γ-ferric oxide (γ-Fe₂O₃).

18. The method for preparing a DTPA-modified ferrofluid according to claim 17, wherein step (a) of mixing an aqueous solution of a magnetic compound with DTPA to form a mixture comprises: (a1) dissolving 4 g to 6 g of ferrous sulfate (FeSO₄) hydrates with 6 to 8 hydration water molecules into 40 to 60 ml of water; (a2) adding 3 g to 5 g of DTPA; and (a3) refluxing and heating at 80° C. to 100° C. for 20 min to 1.5 h.

19. The method for preparing a DTPA-modified ferrofluid according to claim 17, wherein step (b) of adding a basic solution to the mixture comprises: slowly dripping 8 ml to 12 ml of a 10% to 40% sodium hydroxide solution, and refluxing for 1.5 h to 2.5 h.

20. The method for preparing a DTPA-modified ferrofluid according to claim 17, wherein step (c) of taking out of a precipitate from the mixture comprises: taking out of the precipitate by attracting with magnetic force and pouring.

21. A method for preparing a diethylenetriaminepenta acetic acid (DTPA)-modified ferrofluid, wherein the ferrofluid comprises a magnetic compound ferroferric oxide (Fe₃O₄), and the method comprises:
(a) dissolving 3 g to 5 g of ferrous chloride (FeCl₂) hydrates with 3 to 5 hydration water molecules, and 10 g to 13 g of ferric chloride (FeCl₃) hydrates with 5 to 7 hydration water molecules into 100 ml to 200 ml of deoxygenated water, to form a pre-mixture;
(b) refluxing and heating at 80° C. to 100° C., and adding 10 ml to 20 ml 20% to 25% ammonium hydroxide when a temperature of the pre-mixture is raised to 85° C.;
(c) taking a precipitate out from the pre-mixture, and adding glycerol into the precipitate;
(d) adding the precipitate containing glycerol into a solution having a pH of 3 to 5 of N-(2-aminoethyl)-3-aminopropyl-trimethoxysilane (APTES) in deoxygenated water, to form a mixture, and then refluxing and heating the mixture at 80° C. to 100° C. for 2.5 h to 3.5 h;
(e) cooling the mixture to room temperature, adding 2 g to 4 g of DTPA, and then refluxing and heating at boiling temperature for 2.5 h to 3.5 h; and
(f) cooling the mixture to room temperature, and lyophilizing to get a lyophilized semi-finished product.

22. A method for preparing a urea-containing diethylenetriaminepenta acetic acid (DTPA)-modified ferrofluid, comprising: preparing a solution containing a DTPA-modified ferrofluid; and mixing the solution with urea, wherein the DTPA-modified ferrofluid comprises (a) a nano ferrofluid and (b) DTPA at a molar ratio of 90% to 110%.

23. The method for preparing a urea-containing DTPA-modified ferrofluid according to claim 22, wherein the nano ferrofluid is prepared from a magnetic compound selected from γ-ferric oxide (γ-Fe₂O₃) and ferroferric oxide (Fe₃O₄).

24. A method for preparing a peptide ferrofluid, comprising:
(a) preparing a diethylenetriaminepenta acetic acid (DTPA)-modified ferrofluid comprising (1) a nano ferrofluid and (2) DTPA at a molar ratio of 90% to 110%;
(b) adding a peptide to form a peptide ferrofluid pre-product; and
(c) dispersing the peptide ferrofluid pre-product;
wherein a molar ratio of the DTPA-modified ferrofluid to the peptide is greater than 1.0.

25. The method for preparing a peptide ferrofluid according to claim 24, wherein the nano ferrofluid is prepared from a magnetic compound selected from γ-ferric oxide (γ-Fe₂O₃) and ferroferric oxide (Fe₃O₄).

26. The method for preparing a peptide ferrofluid according to claim 24, wherein the peptide is selected from a group consisting of an unmodified peptide and a modified peptide.

27. The method for preparing a peptide ferrofluid according to claim 24, wherein the unmodified peptide is selected from a group consisting of octreotide and lanreotide.

28. The method for preparing a peptide ferrofluid according to claim 24, wherein the modified peptide is selected from a group consisting of DTPA-modified octreotide and DTPA-modified lanreotide.

29. The method for preparing a peptide ferrofluid according to claim 24, wherein step (a) of preparing a DTPA-modified ferrofluid further comprises adding urea.
